(19) United States
(12) Patent Application Publication
(10) Pub. No.: US 2005/0043613 A1
(43) Pub. Date: Feb. 24, 2005

(54) SYSTEM AND METHOD TO REDUCE ARTIFACTS AND IMPROVE COVERAGE IN MR SPECTROSCOPY

(76) Inventors: Eduardo Figueiredo, Sao Paulo (BR); Ricardo Becerra, Waukesha, WI (US); Ceylan C. Guclu, Waukesha, WI (US)

Correspondence Address: ZIOLKOWSKI PATENT SOLUTIONS GROUP, LLC (GEMS), 14135 NORTH CEDARBURG ROAD, MEQUON, WI, 53097, US

(21) Appl. No.: 10/604,832

(22) Filed: Aug. 20, 2003

(51) Int. Cl. A61B 5/05

(52) U.S. Cl. 600/422

(57) ABSTRACT

A system and method of improved homogeneity in internal magnetic resonance (MR) imaging is disclosed. When acquiring MR images that require insertion of an RF coil into a subject, an intracavity coil assembly, or probe, is employed for acquiring MR data of an internal region-of-interest. The intracavity probe includes an RF coil for receiving MR data and a housing enclosing the RF coil. A homogeneity enhancing material is disposable within the housing after insertion into the subject. As a result of disposing the homogeneity enhancing fluid and the RF coil within the housing, inhomogeneities resulting from an air-tissue interface between the RF coil and the subject are reduced.

### BACKGROUND OF INVENTION

The present invention relates generally to magnetic resonance (MR) imaging and, more particularly, to an RF coil embedded with homogeneity enhancing material in a housing and insertable into a patient, such that an improved MR image of a subject may be reconstructed. Specifically, an MR image with reduced artifacts and improved coverage may be reconstructed.

When a substance such as human tissue is subjected to a uniform magnetic field (polarizing field B₀), the individual magnetic moments of the spins in the tissue attempt to align with this polarizing field, but precess about it in random phase at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{Z}, may be rotated, or “tipped”, into the x-y plane to produce a net transverse magnetic moment Mₜ. A signal is emitted by the excited spins after the excitation signal B₁ is terminated and this signal may be received and processed to form an image.

When utilizing these signals to produce images, magnetic field gradients (Gₓ G_{y} and G_{z}) are employed. Typically, the region to be imaged is scanned by a sequence of measurement cycles in which these gradients vary according to the particular localization method being used. The resulting set of received NMR signals are digitized and processed to reconstruct the image using one of many well known reconstruction techniques.

One of the common MR techniques is MR spectroscopy (MRS). Magnetic resonance spectroscopy may be used in vivo for the determination of individual chemical compounds located within a volume of interest. The underlying principle of MRS is that atomic nuclei are surrounded by a cloud of electrons which slightly shield the nucleus from any external magnetic field. As the structure of the electron cloud is specific to an individual molecule or compound, the magnitude of this screening effect is then also a characteristic of the chemical environment of individual nuclei. Since the resonant frequency of the nuclei is proportional to the magnetic field it experiences, the resonant frequency can be determined not only by the external applied field, but also by the small field shift generated by the electron cloud. Detection of this chemical shift, which is usually expressed as “parts per million” (ppm) of the main frequency, requires high levels of homogeneity of the main magnetic field B₀.

Magnetic resonance spectroscopy is particularly useful in the diagnosis and prognosis of various diseases and disorders. For example, MRS is commonly employed as a tool for diagnosis and prognosis of cancer. As an illustration, when examining for prostate cancer, an endorectal coil is employed to insert the RF coil of the MR apparatus within a subject in close proximity to the prostate. By placing the RF coil in close proximity to the area-of-interest the signal-to-noise (SNR) ratio and image resolution are improved.

To secure the coil in close proximity to the area-of-interest and to provide a wider area from which the RF coil may receive MR data, the RF coil is placed in an inflatable housing. Once the RF coil is in place, the inflatable housing and an inflatable retainer are inflated with air, thereby securing the coil's position against the area-of-interest and creating an imaging boundary within the area-of-interest.

However, by surrounding the RF coil with air volumes, imaging can be negatively affected by the tissue-air interface. That is, magnetic flux traveling through the patient that encounters the air volumes react differently from the interactions with the tissue and water of the patient. As a result, the magnetic flux can change direction and negatively affect homogeneity. Specifically, the air-tissue interface can distort the B₀ field and deteriorate the spectral resolution of the area-of-interest that constitutes the air-tissue interface. These susceptibility-induced problems are particularly undesirable in the diagnosis of prostate cancer where approximately 70% of the cancerous tissue is developed along the air-tissue interface.

To alleviate these susceptibility-induced problems, a shimming area is defined so that the air-tissue interface is avoided. As such, distortion of the B₀ field and deterioration of the spectral resolution due to the air-tissue interface are avoided. However, since the shimming area is defined as a box and the area-of-interest is typically not a box, sections of the area-of-interest that are outside the shimming area are missed. Therefore, this solution is again problematic because in the diagnosis of cancer a large amount of the cancerous tissue may lie outside the shimming area. For example, this solution is particularly inadequate in the diagnosis of prostate cancer where approximately 70% of the cancerous tissue is developed along the air-tissue interface and, is therefore, outside the shimming area.

Therefore, when performing MRS utilizing an intracavity probe, which requires inflation, the air-tissue interface can cause information gathered from the MRS scan to be lost or distorted. As such, upon reconstruction of an image, information that is paramount to medical diagnosis may be missing or undecipherable. Particularly when dealing with the diagnosis of diseases such as cancer, missed or undecipherable information can have dire consequences.

It would therefore be desirable to have a system and method of MRS using an intracavity probe without the distortion of the B₀ field and deterioration of the spectral resolution of the area-of-interest due to the air-tissue interface without limiting the imaging area by undersizing a shimming area.

### BRIEF DESCRIPTION OF INVENTION

The present invention provides a system and method of improved magnetic field homogeneity overcoming the aforementioned drawbacks whereby at least a portion of an RF coil assembly is filled with homogeneity enhancing fluid to eliminate an air-tissue interface. As a result of disposing the homogeneity enhancing fluid and the RF coil within a housing, an air-tissue interface, resulting from the inflation of the housing with air is reduced. The present invention is particularly useful in an intracavity coil assembly, or probe, for acquiring MR data of a region-of-interest by inserting the intracavity probe into the patient. However, the advantages achieved by the present invention may be incorporated into other coils for other anatomical regions where susceptibility induced field inhomogeneity needs to be addressed.

Therefore, in accordance with one aspect of the present invention, a probe for improved homogeneity in MR imaging is disclosed that includes an RF coil for receiving MR data and a collapsible housing enclosing the RF coil that is insertable into a subject to be imaged. The collapsible housing is filled with a homogeneity enhancing material after such insertion.

According to another aspect of the present invention, an MR imaging apparatus is disclosed that includes a plurality of gradient coils positioned about a bore of a magnet to impress a polarizing magnetic field, an RF transceiver system, an RF switch controlled by a pulse module to transmit RF signals and an RF coil assembly configured for internal MR image acquisition and having at least one RF coil disposed within an expandable housing to acquire MR images. A homogeneity enhancing fluid is disposed within the housing to improve homogeneity during internal MR image acquisition.

In accordance with a further aspect of the present invention, a method of using an MR imaging device with improved homogeneity is disclosed that includes positioning an RF coil within a housing that is capable of inserted within an imaging subject, and filling the housing with a homogeneity enhancing material.

In yet another aspect of the invention, a kit for an MR imaging device with improved homogeneity is disclosed that includes an RF coil disposed in a flexible housing.

The housing is further configured to be inserted within an imaging subject. A supply of a homogeneity enhancing material is supplied to fill and expand the housing after insertion into the imaging subject.

Various other features, objects and advantages of the present invention will be made apparent from the following detailed description and the drawings.

### BRIEF DESCRIPTION OF DRAWINGS

The drawings illustrate one preferred embodiment presently contemplated for carrying out the invention.

In the drawings:

FIG. 1 is a schematic block diagram of an MR imaging system for use with the present invention.

FIG. 2 is a perspective view of an insertable, intracavity probe in accordance with the present invention and associated pump means.

### DETAILED DESCRIPTION

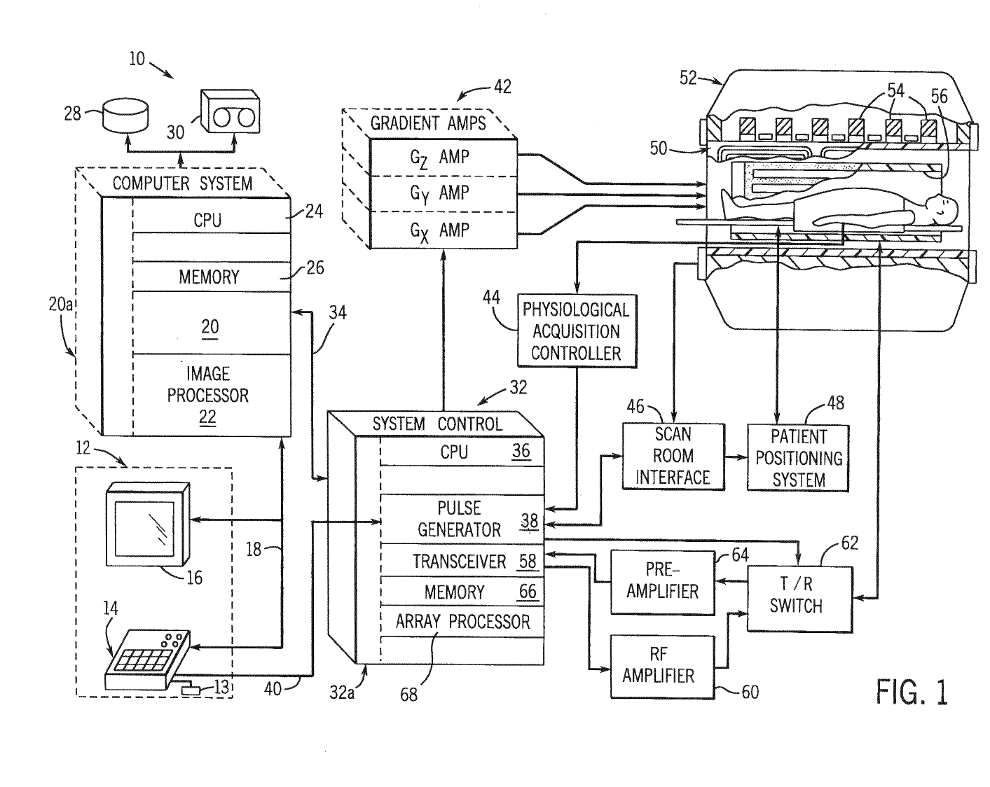

Referring to FIG. 1, the major components of a preferred magnetic resonance imaging (MRI) system 10 incorporating the present invention are shown. The operation of the system is controlled from an operator console 12 which includes a keyboard or other input device 13, a control panel 14, and a display screen 16. The console 12 communicates through a link 18 with a separate computer system 20 that enables an operator to control the production and display of images on the display screen 16. The computer system 20 includes a number of modules which communicate with each other through a backplane 20a. These include an image processor module 22, a CPU module 24 and a memory module 26, known in the art as a frame buffer for storing image data arrays. The computer system 20 is linked to disk storage 28 and tape drive 30 for storage of image data and programs, and communicates with a separate system control 32 through a high speed serial link 34. The input device 13 can include a mouse, joystick, keyboard, track ball, touch activated screen, light wand, voice control, or any similar or equivalent input device, and may be used for interactive geometry prescription.

The system control 32 includes a set of modules connected together by a backplane 32a. These include a CPU module 36 and a pulse generator module 38 which connects to the operator console 12 through a serial link 40. It is through link 40 that the system control 32 receives commands from the operator to indicate the scan sequence that is to be performed. The pulse generator module 38 operates the system components to carry out the desired scan sequence and produces data which indicates the timing, strength and shape of the RF pulses produced, and the timing and length of the data acquisition window. The pulse generator module 38 connects to a set of gradient amplifiers 42, to indicate the timing and shape of the gradient pulses that are produced during the scan. The pulse generator module 38 can also receive patient data from a physiological acquisition controller 44 that receives signals from a number of different sensors connected to the patient, such as ECG signals from electrodes attached to the patient. And finally, the pulse generator module 38 connects to a scan room interface circuit 46 which receives signals from various sensors associated with the condition of the patient and the magnet system. It is also through the scan room interface circuit 46 that a patient positioning system 48 receives commands to move the patient to the desired position for the scan.

The gradient waveforms produced by the pulse generator module 38 are applied to the gradient amplifier system 42 having Gₓ, G_{y}, and G_{z} amplifiers. Each gradient amplifier excites a corresponding physical gradient coil in a gradient coil assembly 50 designated to produce the magnetic field gradients used for spatially encoding acquired signals. The gradient coil assembly 50 forms part of a magnet assembly 52 which includes a polarizing magnet 54 and a whole-body RF coil 56. A transceiver module 58 in the system control 32 produces pulses which are amplified by an RF amplifier 60 and coupled to the RF coil 56 by a transmit/receive switch 62. The resulting signals emitted by the excited nuclei in the patient may be sensed by the same RF coil 56 and coupled through the transmit/receive switch 62 to a preamplifier 64. The amplified MR signals are demodulated, filtered, and digitized in the receiver section of the transceiver 58. The transmit/receive switch 62 is controlled by a signal from the pulse generator module 38 to electrically connect the RF amplifier 60 to the coil 56 during the transmit mode and to connect the coil 56 to the preamplifier 64 during the receive mode. The transmit/receive switch 62 can also enable a separate RF coil (for example, a surface coil) to be used in either the transmit or receive mode.

The MR signals picked up by the RF coil 56 are digitized by the transceiver module 58 and transferred to a memory module 66 in the system control 32. A scan is complete when an array of raw k-space data has been acquired in the memory module 66. This raw k-space data is rearranged into separate k-space data arrays for each image to be reconstructed, and each of these is input to an array processor 68 which operates to Fourier transform the data into an array of image data. This image data is conveyed through the serial link 34 to the computer system 20 where it is stored in memory, such as disk storage 28. In response to commands received from the operator console 12, this image data may be archived in long term storage, such as on the tape drive 30, or it may be further processed by the image processor 22 and conveyed to the operator console 12 and presented on the display 16.

The present invention includes a system and method suitable for use with the above-referenced MR system, or any similar or equivalent system for obtaining MR data. Proton MRS can be implemented on the above-referenced MR system and is used in vivo to measure the concentration of a number of metabolites. High-field magnetic resonance scanners (1.5 T or greater) are typically used to perform MRS studies involving a number of pathologies, including but not limited to various cancers, such as prostate or cervical cancer.

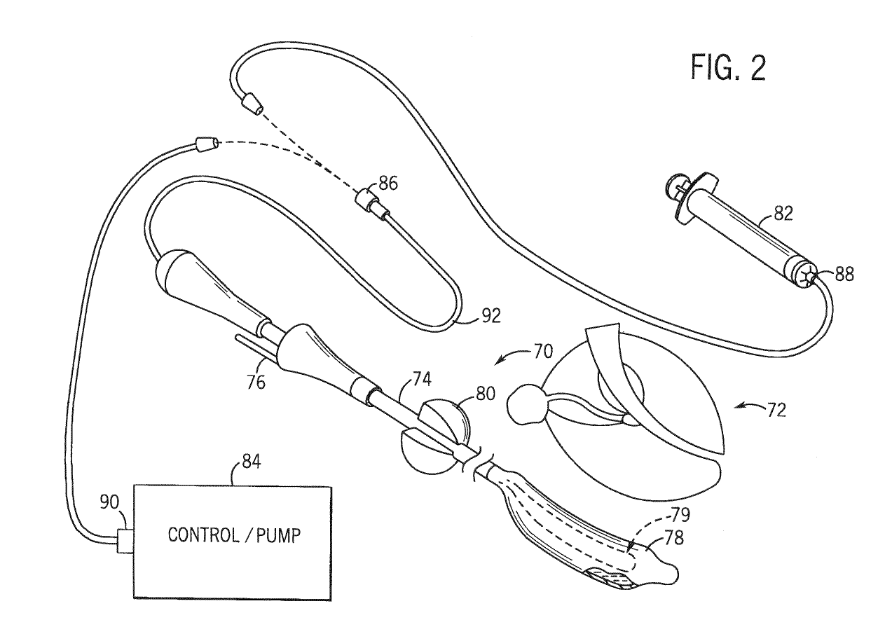

Referring to FIG. 2, an insertable intracavity probe 70 is shown. The insertable intracavity probe 70 is shown operatively engaging a human prostate 72. However, the applicability of the present invention to the examination of the human prostate 72 is for illustrative purposes only and is not intended to limit the applicability of the present invention to imaging of any of a plurality of substances, human or other. Specifically, it is contemplated that the present invention may be utilized in conjunction with MR imaging of any substance that requires insertion of the RF coil into an orifice. For example, the present invention may be utilized when imaging requires insertion of the RF coil into the rectum, vagina, mouth or any orifice, or surgical incision.

The insertable intracavity probe 70 is an MRI or NMR receiving device capable of receiving MR data. The insertable intracavity probe 70 is capable of transmitting RF excitation for MR imaging. The probe 70 includes a hollow shaft 74 that extends from a handle 76 to a collapsible housing 78, preferably an expandable membrane. An MR RF coil 79 is disposed in the expandable membrane 78. The RF coil contained within the probe 70 is electrically connected to an MR imaging system of the type shown if FIG. 1. Still referring to FIG. 2, the handle 76 provides a means of accurately positioning the expandable membrane 78 within imaging proximity of the prostate 72. A retainer 80 is provided to secure the probe against migration after insertion and positioning of the RF coil. It is contemplated that the retainer 80 may be solid or inflated along with the expandable membrane 78, as will be described.

Once the probe 70 is positioned such that the RF coil is within proximity of the prostate 72, a homogeneity enhancing material is pumped from a reservoir, such as syringe 82 or electronically controlled pump 84, to the expandable membrane 78. Specifically, the probe 70 is connected to a pump by interface 86, which is configured to operatively engage nozzle 88 of syringe 82 or outlet 90 of electronically controlled pump 84. Once connected the homogeneity enhancing material is pumped through supply tube 92 to the hollow shaft 74. The homogeneity enhancing material is forced down the hollow shaft 74 and into the expandable membrane 78. As a result, the expandable membrane 78 inflates, thereby pressing the expandable membrane about a portion of the prostate 72. Accordingly, the RF coil, disposed within the expandable membrane 78, is positioned within ideal proximity of the prostate 72. The expandable membrane 78 contacts the prostate 72 and impresses about the prostate 72. As a result, a wider imaging area is achieved. Furthermore, the homogeneity enhancing material may also be utilized to inflate the retainer 80 along with inflating the expandable membrane 78, if the retainer 80 is inflatable.

By selectively pumping the homogeneity enhancing material through the probe 70 to the expandable membrane 78, several different anatomical regions can be imaged without requiring different coils. The syringe 82 allows manual control of the inflation of the expandable membrane 78. On the other hand, the electronically controlled pump 84 can be configured to automatically inflate the expandable membrane until sufficient inflation is sensed. Also, the location as well as the degree of homogeneity enhancement may be controlled simply by controlling the amount of homogeneity enhancing material that is pumped into the expandable membrane 78 and the specific concentration of the homogeneity enhancing material. If the electronically controlled pump 84 is being used, the electronically controlled pump 84 can automatically augment the degree of homogeneity enhancement in response to feedback.

In a preferred embodiment, the homogeneity enhancing material is a fluorocarbon. Fluorocarbons have magnetic permeability properties similar to that of human tissue and are highly effective in preventing field inhomogeneity. Specifically, hydrogen-depleted fluorocarbons have magnetic susceptibility properties similar to that of human tissue and since they have low hydrogen content, they do not contribute any signal to the MR image.

Perfluorocarbon, such as FC-77, is particularly well suited for disposal within the expandable membrane 78 as its high electrical resistivity and its low dielectric constant allows the material to be placed within the membrane without affecting the performance of the RF coil. A number of other perfluorocarbons may be utilized such as FC-87, FC-72, FC-84, FC-3283, FC-40, FC-43, and FC-70. In a preferred embodiment, the perfluorocarbon is in the form of either a liquid or a gel. The perfluorocarbon is pumped into the expandable membrane 78 to cause the expandable membrane to expand about the area-of-interest and thereby eliminate the need to inflate the expandable membrane with air. As such, the air-tissue interface is eliminated. These compounds are also used as artificial blood substitute, and are non-toxic, i.e safe in the case of leakage.

The characteristics of these perfluorocarbons are such that it may also serve to cool hotspots (areas of high temperature) on the RF coil. As such, the homogeneity enhancing material may act as a heat sink, thereby absorbing heat from the coil and dispersing the heat across a larger area. Such heat dispersion can be particularly important when the probe 70 is disposed within the patient, such as when the probe is an endorectal probe.

It is contemplated that the invention described above may be embodied in a probe for improved homogeneity in MR imaging. The probe includes an RF coil for receiving MR data, a housing enclosing the RF coil and insertable into a subject to be imaged, and a homogeneity enhancing material disposable within the housing.

It is further contemplated that the above described invention may be embodied with an MR imaging apparatus. The MR imaging apparatus includes a plurality of gradient coils positioned about a bore of a magnet to impress a polarizing magnetic field, an RF transceiver system, and an RF switch controlled by a pulse module to transmit RF signals. An RF coil assembly is configured for internal MR image acquisition and has at least one RF coil disposed within an expandable housing to acquire MR images. A homogeneity enhancing fluid is provided for expansion of the expandable housing to improve homogeneity during internal MR image acquisition.

It is also contemplated that the above described invention may be embodied as a method of using an MR imaging device with improved homogeneity that includes positioning an RF coil within a housing. The housing is capable of being inserted within he subject being imaged (examined.) The process also includes filling the housing with a homogeneity enhancing material.

A kit is also contemplated that has an MR imaging device with improved homogeneity. The kit includes an RF coil disposed in a flexible housing. The housing is further configured to be inserted within an imaging subject. A supply of a homogeneity enhancing material is supplied to fill and expand the housing after insertion into the imaging subject.

The present invention has been described in terms of the preferred embodiment, and it is recognized that equivalents, alternatives, and modifications, aside from those expressly stated, are possible and within the scope of the appending claims.

1. A probe for improved homogeneity in magnetic resonance (MR) imaging, the probe comprising:
an RF coil for receiving MR data;
a collapsible housing enclosing the RF coil and constructed for insertion into a subject to be imaged; and
a homogeneity enhancing material disposable within the collapsible housing.

2. The probe of claim 1 wherein the homogeneity enhancing material has a magnetic permeability similar to that of the subject.

3. The probe of claim 1 wherein the collapsible housing is an expandable membrane to allow the RF coil to receive MR data from a wider area of the subject when expanded with the homogeneity enhancing material then would be otherwise.

4. The probe of claim 1 wherein the homogeneity enhancing material includes one of a gel and a liquid.

5. The probe of claim 1 wherein the homogeneity enhancing material includes a material having a magnetic permeability similar to that of water.

6. The probe of claim 1 wherein the homogeneity enhancing material includes a perfluorocarbon material.

7. The probe of claim 3 wherein the homogeneity enhancing material expands the collapsible housing after insertion into the subject to be imaged.

8. The probe of claim 7 wherein the collapsible housing is constructed free of gases.

9. The probe of claim 1 formed as an endorectal probe.

10.The probe of claim 1 further comprising an inflatable retainer that secures the RF coil within the subject to be imaged when inflated with the homogeneity enhancing fluid.

11. An MR imaging apparatus comprising:
a plurality of gradient coils positioned about a bore of a magnet to impress a polarizing magnetic field;
an RF transceiver system;
an RF switch controlled by a pulse module to transmit RF signals;
an RF coil assembly configured for internal MR image acquisition and having at least one RF coil disposed within a housing that is constructed for insertion into a subject; and
a homogeneity enhancing fluid disposable within the housing to improve homogeneity during internal MR image acquisition.

12. The MR imaging apparatus of claim 11 wherein the housing is an expandable membrane and the homogeneity enhancing fluid causes the expandable membrane to inflate.

13. The MR imaging apparatus of claim 11 wherein the homogeneity enhancing material has a magnetic permeability similar to that of the subject.

14. The MR imaging apparatus of claim 11 wherein the homogeneity enhancing fluid includes a perfluorocarbon material.

15. The MR imaging apparatus of claim 11 further comprising a control means to inflate the housing with the homogeneity enhancing fluid.

16. The MR imaging apparatus of claim 15 wherein the control means comprises a syringe.

17. The MR imaging apparatus of claim 15 wherein the control means comprises an electronically controlled pump.

18. The MR imaging apparatus of claim 15 wherein the control means automatically inflates the housing with the homogeneity enhancing fluid.

19. The MR imaging apparatus of claim 11 further comprising a retainer filled with the homogeneity enhancing fluid to secure the at least one RF coil within the subject.

20. A method of using an MR imaging device with improved homogeneity comprising:
positioning an RF coil within a housing that is capable of being inserted within an imaging subject; and
filling the housing with a homogeneity enhancing material.

21. The method of manufacturing of claim 20 further comprising attaching a pump to the housing to inflate the housing with the homogeneity enhancing material.

22. The method of manufacturing of claim 20 further comprising attaching an automated inflation control to control the inflation the housing with a homogeneity enhancing material.

23. The method of manufacturing of claim 20 wherein the homogeneity enhancing material comprises a perfluorocarbon material.

24. The method of manufacturing of claim 20 wherein the homogeneity enhancing material includes one of a gel and a liquid.

25.The method of manufacturing of claim 20 wherein the homogeneity enhancing material has a magnetic permeability similar to that of the imaging subject.

26. A kit for an MR imaging device with improved homogeneity comprising:
an RF coil;
a flexible housing configured to contain the RF coil therein and further configured to be inserted within an imaging subject; and
a supply of a homogeneity enhancing material to fill and expand the flexible housing after insertion into the imaging subject.
